(19) United States
(12) Patent Application Publication
(10) Pub. No.: US 2003/0148471 A1
(43) Pub. Date: Aug. 7, 2003

(54) Method for producing maltose syrup by using a hexosyltransferase

(76) Inventors: Barrie Edmund Norman, Birkerod (DK); Hanne Vang Hendriksen, Holte (DK)

Correspondence Address: NOVOZYMES NORTH AMERICA, INC., 500 FIFTH AVENUE, SUITE 1600, NEW YORK, NY, 10110, US

(21) Appl. No.: 10/332,939

(22) PCT Filed: Jul. 17, 2001
(86) PCT No.: PCT/DK01/00504

(30) Foreign Application Priority Data
Jul. 28, 2000 (DK) PA200001147
Aug. 17, 2000 (US) 60225037

(51) Int. Cl. C12P 19/18; C12P 19/12

(52) U.S. Cl. 435/97; 435/100

(57) ABSTRACT

The present invention relates to a method of producing maltose syrup, wherein starch is treated with a hexosyltransferase (E.C. 2.4.1) and then a beta-amylase and/or a maltogenic amy-lase, or variant thereof. The invention also relates to an intermediate product suitable as staring material in maltose production processes.

The present invention concerns a method of production of maltose syrup, whereby starch is treated with a hexosyltransferase and a beta-amylase and/or a maltogenic amylase, or variant thereof. Further, the invention also relates to a suitable starting material for the production of maltose.

### BACKGROUND OF THE INVENTION

Maltose

Maltose is a disaccharide having the chemical structure of 4-O-alpha-D-glucopyronosyl-D-glucopyranose (C₁₂H₂₂O₁₁) and is the main component of maltose syrups.

Application of Maltose Syrups

Maltose is used to replace sucrose in a number of foodstuffs and confectionary products as well as starting product for hydrogenation to maltitol.

Maltose does not crystallize easily, in contrast to, e.g., glucose, which is able to crystallize even in the presence of impurities in high concentrations. Maltose is not able to crystallize and thus to be purified further, unless the maltose used as a starting material exhibits a purity above 90%. Also, the fact that maltose does not crystallize easily is one of the reasons why maltose is a valuable raw material in the candy industry.

Maltose has also other applications, e.g., as the active component of intravenous injection liquids intended for provision of sugar for the patient and as a component in frozen deserts (due to low crystallization ability of maltose), in the baking and brewing industry, and for production of maltitol, which can be used as a sweetening agent, like sorbitol, vide Glycose Sirups, Science and Technology, Elsevier Applied Science Publishers 1984, pages 117-135.

Thus, the purpose of the invention is to provide a method of producing maltose syrup.

### DESCRIPTION OF THE INVENTION

The object of the present invention is to provide a method of producing high maltose syrup.

At least in the context of the present invention high maltose syrup is syrup with a maltose content of at least 80%, preferably at least 90%.

Traditional Methods of Producing Maltose

The starting (raw) material for maltose production processes is starch from corn, potato, sweet potato, manioc, rice, and cassava in a concentration of about 10-20% for production of medical grade maltose and in the range of 20-40% for food grade maltose.

Various methods of producing maltose have been known and the purity of the maltose obtained by these methods is in the range from 75-90%. A typical example of these methods comprise heating and treating a starch slurry with alpha-amylase thereby liquefying the starch and then hydrolysing the liquefied starch with beta-amylase and pullulanase (alpha-1,6-glucosidase) to afford a maltose solution, which may then be purified.

The greater part of the “impurities” in this maltose solution comprises glucose and oligosaccharides, such as maltotriose and limit dextrins, which consists of three or more glucose units.

Method of the Invention

The inventors of the present invention have found, that a maltose syrup with a higher maltose content can be obtained—than when liquefying with an alpha-amylase-by treating a starch slurry with a hexosyltransferase (E.C. 2.4.1) followed by treatment with a beta-amylase (E.C. 3.2.1.2) and/or a maltogenic amylase, or a variant thereof.

Thus, in the first aspect the invention relates to a method of producing maltose, wherein starch is treated with

i) a hexosyltransferase (E.C. 2.4.1), and then

ii) a beta-amylase and/or a maltogenic amylase, or a variant thereof.

Preferably the method of the invention comprises the steps of:

a) treating starch with a hexosyltransferase (E.C. 2.4.1) until a product having

i)an Additional Degree of Branching (ADB) of between 10-150% has been provided if using a branching enzyme, and/or

ii) a viscosity corresponding to that of liquefied starch obtainable by treating 30% DS starch with wild type Bacillus licheniformis alpha-amylase until a DE in the range from 8-15, preferably DE of between 10-12, has been provided,

b) the product provided in step a) is treated with a beta-amylase and/or a maltogenic amylase, or variant thereof, and: optionally

c) recovering and/or purifying maltose from the product provided in step b).

Dextrose Equivalent (DE) and Additional Degree of Branching (ADB)

The degree of branching of a starch product is determined by measuring the DE (Dextrose Equivalent) of the starch product after debranching with isoamylase (Pseudomonas isoamylase available from Hayashibara, Japan). The higher the DE (after debranching), the higher the degree of branching. The additional degree of branching (ADB) introduced by the hexosyltransferase is calculated as

$$Additional Degree of Branching = \frac{DE - X}{X} \times 100 \%$$

X is the DE after debranching of native starch (i.e., the starting material).

Method for Treating Starch with Hexosyltransferase

To obtain an effective enzymatic treatment of the starch, the starch may in a preferred embodiment be gelatinised. Dependent upon the heat stability of the enzyme and the gelatinisation temperature of the specific starch this can be done either in combination with enzyme treatment or prior to enzyme treatment. Gelatinisation may be carried-out either in a batch system or continuously in a jet-cooker. In lab-scale simple heating systems can be used, e.g., an oil bath, pressure cooker or autoclave. Specific reaction conditions for the subsequent enzymatic treatment or for the combined gelatinisation/enzyme process, i.e., temperature, pH, % DS, dosage and incubation time depend upon the characteristics of the enzyme and the starch source.

In an embodiment the starch slurry in step a) of the process of the invention is treated at 50-150° C., preferably in the range from 50-100° C. In the case of using a 1,4-alpha-glucan branching enzyme as the hexosyltransferase the treating temperature will be in the range from 50-70° C., preferably around 65° C. In an embodiment, when using 1,4-alpha-glucan branching enzyme the starch is gelatinized first by jetting at 105° C.-140° C. and then cooled to about 65° C.

In the case of 4-alpha-glucanotransferase (amylomaltases or D-enzyme) and CGTase a gelatination step is optional and may be left out. Thus, starch may be treated (incubated directly with the enzyme(s)) at from 80-100° C., preferably around 90° C.

A pullulanase may be added in step b). In general the starch (raw) starting material is a 10-50% DS starch slurry, preferably a 20-40% DS starch slurry, especially a starch slurry having around 30% DS.

The invention also relates to a product obtainable by

a) treating starch with a hexosyltransferase (E.C. 2.4.1)

i)until a product having an Additional Degree of Branching (ADB) of between 10-150% has been provided when using a branching enzyme, and/or

ii) a viscosity corresponding to that of liquefied starch obtainable by treating 30% DS starch with wild type Bacillus licheniformis alpha-amylase until a DE in the range from 8-15, preferably DE of between 10-12, has been provided.

The product is suitable as starting material for maltose processes. The product is stable and have a reduced tendency to retrograde and is soluble in aqueous solution.

In a further aspect the invention relates to a maltose-containing product obtainable by the method of the invention. The product obtained this way has a maltose (DP₂) content of above 80%, preferably 85%, especially above 90%.

Further, the invention also relates to the use of the product of the invention prepared by treating starch with a hexyltransferase as starting (raw) material (substrate) for the production of maltose.

Hexosyltransferase

Examples of hexyltransferase (E.C. 2.4.1) include 1,4-alpha-Glucan branching enzyme (E.C. 2.4.1.18); 4-alpha-glucanotransferase (amylomaltases or D-enzyme) (E.C. 2.4.1.25); Cyclomaltodextrin glucanotransferase (CGTase) (E.C. 2.4.1.19).

1,4-alpha-glucan branching enzyme

In an embodiment the hexosyl transferease is 1,4-alpha-glucan branching enzyme which forms additional 1,6-glucosidic linkages of amylopectin and converts amylose into amylopectin. Amylopectin branching enzyme is frequently termed Q-enzyme.

A specifically contemplated 1,4-alpha-Glucan branching enzyme is derived from Rhodothermus, preferably Rhodothermus obamensis, especially the deposited strain E. coli DSM 12607 comprising the glgB gene from Rhodothermus obamensis.

4-alpha-igucanotransferase (amylomaltases or D-enzyme)

In an embodiment the hexosyl transferease is 4-alpha-glucanotransferase, which transfers a segment of a 1,4-alpha-D-glucan to a new 4-position in an acceptor, which may be glucose or 1,4-alpha-D-glucan. 4-alpha-glucanotransferase is often referred to as D-enzyme.

A specifically contemplated 4-alpha-glucanotransferase is derived from Thermococcus litoralis (Jeon, Beong-Sam, et al: 4-alpha-Glucanotransferase from the hyperthermophilic archaeon Thermococcus litoralis (1997). Eur.J.Biochem. 248: 171-178.)

CGTase

In an embodiment the CGTase or CGTase variant is derived from a strain of Bacillus, a strain of Brevibacterium, a strain of Clostridium, a strain of Corynebacterium, a strain of Klebsiella, a strain of Micrococcus, a strain of Therxmoanaerobium, a strain of Thermoanaerobacter, a strain of Thermoanaerobacterium, a strain of Thermoanaerobacterium, or a strain of Thermoactinomyces.

In a preferred embodiment, the CGTase or CGTase variant is derived from a strain of Bacillus autolyticus, a strain of Bacillus cereus, a strain of Bacillus circulans, a strain of Bacillus circulans var. alkalophilus, a strain of Bacillus coagulans, a strain of Bacillus firmus, a strain of Bacillus halophilus, a strain of Bacillus macerans, a strain of Bacillus megateriurn, a strain of Bacillus ohbensis, a strain of Bacillus stearothermophilus, a strain of Bacillus subtilis, a strain of Klebsiella pneumonia, a strain of Thermoanaerobacter ethanolicus, a strain of Thermoanaerobacter finnii, a strain of Clostridium thermoamylolyticum, a strain of Clostridium thermosaccharolyticum, or a strain of Thermoanaerobacterium thermosulfurigenes.

In a more preferred embodiment the CGTase or CGTase variant is derived from the strain of Bacillus sp. strain 1011, the strain Bacillus sp. strain 38-2, the strain Bacillus sp. strain 17-1, the strain Bacillus sp. 1-1, the strain Bacillus sp. strain B1018, the strain Bacillus circulans Strain 8, the strain Bacillus circulans Strain 251, or the strain Therrmoanaerobacter sp. ATCC 53627, or mutants or variants thereof.

Contemplated CGTase variants are disclosed in WO 96/33267 (Novozymes A/S) and WO 99/15633 (Novozymes A/S), which are hereby incorporated by reference.

A commercially available CGTase product is Toruzyme® from Novozymes A/S.

Beta-Amylase

Examples of beta-amylases (E.C. 3.2.1.2) include crop beta-amylases, such as barley beta-amylases, wheat beta-amylases, soybean beta-amylases, and beta-amylases derived from Bacillus sp., such as the beta-amylase disclosed in U.S. Pat. No. 4,970,158 (Novozymes A/S).

Commercially available beta-amylases include Spezyme® BBA and Spezyme® DBA from Genencor Int.

Pullulanase

Examples of pullulanases (E.C. 3.2.1.41) include a thermostable pullulanase from, e.g., Pyrococcus or a protein engineered pullulanase from, e.g., a Bacillus strain such as Bacillus acidopullulyticus or Bacillus deramificans (e.g., the Bacillus deramificans pullulanase with GeneBank accession number Q68699).

Commercially available pullulanases include Promozyme® D from Novozymes A/S and Optimax® from Genencor Int.

Maltogenic Amylase

A maltogenic amylase (E.C. 3.1.133) hydrolyses 1,4-alpha-D-glucosidic linkages in polysaccharides so as to remove successive alpha-maltose residues from the non-reducing ends of the chains. A maltogenic amylases acts on starch and related polysaccharides and oligosaccharides.

An Example of a suitable maltogenic amylase is the maltogenic amylase produced by Bacillus stearothermophilus C599 disclosed in EP patent no. 120,693 (Novo Industri A/S). Contemplated maltogenic amylase variants are disclosed in WO 99/43794 and WO 99/43793, which are thereby incorporated by reference.

A commercially available maltogenic amylase is sold under the tradename Maltogenase® (Novozymes A/S, Denmark).

### MATERIALS & METHODS

Branching enzyme: Rhodothermus obamensis D-enzyme disclosed in WO 00/58445 (Novozymes A/S, Denmark).

Spezyme® BBA 1500 is a barley beta-amylase (available from Genencor Int., USA).

Promozyme® D is a pullulanase derived from a strain of Bacillus (available from Novozymes A/S, Denmark)

Isoamylase: Pseudomonas isoamylase (available from Hayashibara, Japan).

Toruzyme® is a heat-stable cyclomaltodextrin glucanotransferase (CGTase) derived from a strain of Thermoanaerobacter (available on request from Novozymes A/S, Denmark). Termamyl® LC is an alpha-amylase derived from Bacillus licheniformis (available on request from Novozymes A/S, Denmark)

Maltogenase® (available from Novozymes A/S, Denmark). Wild type Bacillus licheniformis alpha-amylase is disclosed as SEQ ID NO: 4 in WO 99/19467. (Novozymes A/S, Denmark).

Methods

Beta-amylase Activity (DP°)

The activity of Spezyme® BBA 1500 is expressed in Degree of Diastatic Power (DP°). It is the amount of enzyme contained in 0.1 ml of a 5% solution of the sample enzyme preparation that will produce sufficient reducing sugars to reduce 5 ml of Fehling's solution when the sample is incubated with 100 ml of substrate for 1 hour at 20° C. (68° F.).

Pullulanase Activity (New Pullulanase Unit Novo (NPUN)

One new Pullulanase Unit Novo (NPUN) is a unit of endo-pullulanase activity and is measured relative to Novozymes standard made on 0.7% Red Pullulan, 40° C., pH 4.5, 30 minutes reaction time. A detailed description of the analysis method is available on request (SOP No.: EB-SM.0420.02/01).

Branching Enzyme Activity

Branching Enzyme activity is determined according to a modified version of the procedure described by Takata et al., Applied and Environmental Microbiology (1994), p. 3097 (assay A):

20 micro 1 enzyme solution is mixed with 50 micro litre water and 50 micro litre substrate solution and incubated for 30 minutes at testing temperature. The substrate solution is 0.1% type III amylose dissolved in Tris buffer. The reaction is terminated by the addition of 2 ml of iodine reagent. Iodine reagent is made daily from 0.5 ml of stock solution (0.26 g of I₂ and 2.6 g of KI in 10 ml of water) mixed with 0.5 ml of 1 N HCl and diluted to 130 ml. The mixture is incubated for 15 minutes at room temperature to stabilize the colour. Activity is measured as difference in A660 between a tested sample and a control in which cell extract is replaced by water. One unit of branching enzyme activity is defined as the amount of enzyme that can decrease the A660 of the amylose-iodine complex by 1% per minute at 60° C., pH 7.0.

Method for Treating Starch with Hexosyltransferase

a) when using Branching Enzyme the following procedure may be used:

A 10-30% DS, pH 7-8, starch suspension is gelatinised in a jet-cooker or an autoclave. The slurry is then cooled to 60-70° C. and branching enzyme added. When the specified conversion is reached (total time will depend upon dosage) the reaction is terminated by heating to 100° C. for 15 minutes.

b) for Glucanotransferase or CGT′ ase preparation can be made as follows:

A 30% DS, pH 5-7, starch suspension is prepared and enzyme added. The suspension is then heated to 70-90° C. and incubated for 4-24h. When the specified conversion is reached (total time will depend upon dosage) the reaction is terminated by heating to 140° C. for 5-15 minutes.

### EXAMPLES

### Example 1

Production of Maltose

30% DS potato starch substrate liquefied with Branching Enzyme was prepared. The starch slurry was clear and stable. The starch slurry was then treated with beta-amylase (1 DP°/g DS) and pullulanase (1 NPUN/g DS) at 60° C., pH 5.0.

After 72 hours incubation 89% maltose was obtained. In comparison to this only 75% maltose was obtained under identical conditions using Termamyl LC DE 10 maltodextrin.

1. A method of producing maltose, wherein starch is treated with
i) a hexosyltransferase (E.C. 2.4.1), and then
ii) a beta-amylase (E.C. 3.2.1.2) and/or a maltogenic amylase (E.C. 3.2.1.133), or variant thereof.

2. The method of claim 1, comprising the steps of:
a) treating starch with a hexosyltransferase (E.C. 2.4.1) until a product having
i) an Additional Degree of Branching (ADB) of between 10-150% has been provided if using a branching enzyme, and/or
ii) a viscosity corresponding to that of liquefied starch obtainable by treating 30% DS starch with wild type Bacillus licheniformis alpha-amylase until a DE in the range from 8-15, preferably DE of between 10-12, has been provided,
b) the product provided in step a) is treated with a beta-amylase and/or a maltogenic amylase, or variant thereof, and optionally
c) recovering and/or purifying maltose from the product provided in step b).

3. The method of claim 2 wherein starch in step a) is treated at 50-150° C., preferably in the range from 50-100° C.

4. The method of claim 1 or 2, wherein further a pullulanase is added in step b).

5. The method of any of claims 1-4, wherein a 10-50% DS starch slurry, preferably a 20-40% DS, especially around 30% DS starch slurry is used as the starting material.

6. The method of claim 1, wherein the hexyltransferase (E.C. 2.4.1.) is one or more of the enzymes selected from the group consisting of 1,4-alpha-Glucan branching enzyme (E.C. 2.4.1.18); 4-alpha-glucanotransferase (amylomaltases or D-enzyme) (E.C. 2.4.25); Cyclomaltodextrin glucanotransferase (CGTase) (E.C. 2.4.1.19).

7. The method of claims 1-6, wherein the starch in step a) is treated with a combination of D-enzyme and a maltogenic amylase or variant thereof.

8. The method of claim 1, wherein the beta-amylase (E.C. 2.4.1.2) is barley beta-amylase.

9. The method of claim 1, wherein the pullulanase is a Bacillus pullulanase.

10. The method of claim 6, wherein the 1,4-alpha-Glucan branching enzyme (E.C. 2.4.1.18) is derived from Rhodotherimus, preferably Rhodothermus obamensis.

11. The product obtainable by treating starch with a hexosyltransferase (E.C. 2.4.1) until a product having
i) an Additional Degree of Branching (ADB) of between 10-150% has been provided if using a branching enzyme, and/or
ii) a viscosity corresponding to that of liquefied starch obtainable by treating 30% DS starch with wild type Bacillus licheniformis alpha-amylase until a DE in the range from 8-15, preferably DE of between 10-12, has been provided.

12. The product of claim 11, wherein the hexoyltransferase (E.C. 2.4.1) is one or more of the enzymes selected from the group consisting of 1,4-alpha-Glucan branching enzyme (E.C. 2.4.1.18); 4-alpha-glucanotransferase (amylomaltases or D-enzyme) (E.C. 2.4.25); Cyclomaltodextrin glucanotransferase (CGTase) (E.C. 2.4.1.19).

13. The method of claim 12, wherein the 1,4-alpha-Glucan branching enzyme (E.C. 2.4.1.18) is derived from Rhodothermus, preferably Rhodothermus obamensis, especially the deposited strain E. coli DSM 12607 comprising the glgB gene from Rhodothermus obamensis.

14. The syrup product obtainable by the method of any of claims 1-10.

15. The syrup product of claim 14, wherein the maltose (DP₂) content is above 80%, preferably 85%, especially above 90%.

16. Use of a product of any of claims 11-13, as starting material (substrate) for the production of maltose.

17. Use according to claim 16, wherein a portion of the product is used as starting material (substrate) for the production of maltose.
